(19) United States
(12) Patent Application Publication
(10) Pub. No.: US 2014/0107457 A1
(43) Pub. Date: Apr. 17, 2014

(54) SYSTEM AND METHOD OF DIAGNOSING AN ELECTROCARDIOGRAM (ECG) SENSING SYSTEM

(71) Applicant: CYBERONICS, INC., Houston, TX (US)

(72) Inventor: Shriram RAGHUNATHAN, Houston, TX (US)

(73) Assignee: CYBERONICS, INC., Houston, TX (US)

(21) Appl. No.: 13/653,510

(22) Filed: Oct. 17, 2012

(51) Int. Cl. A61B 5/0402; A61B 5/0408

(52) U.S. Cl. 600/386; 600/509

(57) ABSTRACT

An electrocardiogram (ECG) sensor system is disclosed that includes one or more interface connectors configured to be coupled to one or more electrodes. The ECG sensor system includes a processor configured to receive an ECG signal via the one or more interface connectors. The processor may be configured to determine one or more noise values associated with the ECG signal. Each of the one or more noise values may be indicative of a measurement of a component of the ECG signal that is potentially related to a non-heart beat source. The processor may be configured to send one or more noise values to an automated troubleshooting system.

### FIELD OF THE DISCLOSURE

The present disclosure is generally related to medical device diagnostics.

### BACKGROUND

An electrocardiogram (ECG) measures electrical activity of a patient's heart. An ECG may be used to measure rate and regularity of heart beats of the heart. ECG data may be obtained from a patient by one or more electrodes attached to an exterior surface (e.g., skin) of the patient. Heart rate activity and ECG data may be used to detect onset or occurrence of a variety of medical conditions, such as seizures. Seizures may be characterized by episodes of disturbed brain activity that cause changes in attention or behavior. Increased heart rate and changes in ECG data may be correlated to an onset or an occurrence of a seizure.

However, detection of an occurrence of or an onset of a seizure may be difficult to monitor given that seizures may be unpredictable and that monitoring relies on self-reporting by a patient. A patient may record information during an occurrence of a seizure, but the patient's record of a seizure may provide limited diagnostic information as to specific measurements of ECG data. In some situations, a patient may be asleep during a seizure event, and as such is unable to log seizure activity. Many seizure prone patients are pediatric patients, who may not be able to accurately detect or report seizure events given their age and abilities to distinguish symptoms of a seizure event.

Patients who are prone to seizures may be monitored regularly using automated detectors that are capable of monitoring diagnostic information used to detect seizures. However, such automated detectors may be prone to errors based on user error, daily use, and failures in the automated device. Further, a patient may require assistance from a physician or a technician to troubleshoot problems with the automated detector, thereby causing inconvenience to the patient.

### SUMMARY

In a particular embodiment, a method includes sensing electrocardiogram (ECG) data at an ECG sensor system. The method further includes processing the sensed ECG data at the ECG sensor system to determine one or more noise values, where each noise value is indicative of a measurement of a component of the sensed ECG data that is potentially related to a non-heart beat source. The method further includes sending the one or more noise values to an automated troubleshooting system to troubleshoot the ECG sensor system.

In another particular embodiment, an ECG sensor system includes one or more interface connectors configured to be coupled to one or more electrodes. The ECG sensor system includes a processor configured to receive an ECG signal via the one or more interface connectors. The processor may be configured to determine one or more noise values associated with the ECG signal. Each of the one or more noise values may be indicative of a measurement of a component of the ECG signal that is potentially related to a non-heart beat source. The processor may be configured to send one or more noise values to an automated troubleshooting system.

In another particular embodiment, a computer-readable medium includes instructions that, when executed by a processor cause the processor to sense electrocardiogram (ECG) data at an ECG sensor system. The instructions may be further configured to cause the processor to process the sensed ECG data at the ECG sensor system to determine one or more noise values, where each noise value is indicative of a measurement of a component of the sensed ECG data that is potentially related to a non-heart beat source. The instructions may be further configured to cause the processor to send the one or more noise values to an automated troubleshooting system to troubleshoot the ECG sensor system.

In another particular embodiment, a method includes receiving one or more noise values from an ECG sensor system at an automated troubleshooting system. Each noise value of the one or more noise values may be indicative of a measurement of a component of an ECG signal sensed by the ECG sensor system. The measurement of the component may be potentially related to a non-heart beat source. The method further includes performing a determination, based on the one or more noise values, of whether ECG data corresponding to the sensed ECG signal satisfies one or more criteria. The method further includes generating an output based on the determination.

In another particular embodiment, an automated troubleshooting system includes a receiver to receive one or more noise values from an ECG sensor system. Each noise value of the one or more noise values may be indicative of a measurement of a component of an ECG signal sensed by the ECG sensor system that is potentially related to a non-heart beat source. The automated troubleshooting system may include a processor coupled to the receiver. The processor may be configured to perform a determination, based on the one or more noise values, of whether ECG data corresponding to the ECG signal satisfies one or more criteria. The processor may be configured to generate an output based on the determination.

In another particular embodiment, a computer-readable medium includes instructions that, when executed by a processor cause the processor to receive one or more noise values from an ECG sensor system at an automated troubleshooting system. Each noise value of the one or more noise values may be indicative of a measurement of a component of an ECG signal sensed by the ECG sensor system. The measurement of the component may be potentially related to a non-heart beat source. The instructions may be further configured to cause the processor to perform a determination, based on the one or more noise values, of whether ECG data corresponding to the sensed ECG signal satisfies one or more criteria. The instructions may be further configured to cause the processor to generate an output based on the determination.

In another particular embodiment, a system for diagnosing an ECG sensor system includes the ECG sensor system and an automated troubleshooting system. The ECG sensor system includes a first processor configured to receive an ECG signal via one or more interface connectors. The first processor may be configured to determine one or more noise values associated with the ECG signal. Each of the one or more noise values may be indicative of a measurement of a component of the ECG signal that is potentially related to a non-heart beat source. The first processor may be configured to send one or more noise values to an automated troubleshooting system. The automated troubleshooting system may be configured to receive the one or more noise values from the ECG sensor system. The automated troubleshooting system may include a second processor configured to perform a determination, based on the one or more noise values, of whether ECG data corresponding to the ECG signal satisfies one or more criteria. The second processor may be configured to generate an output based on the determination.

The features, functions, and advantages of the disclosed embodiments can be achieved independently in various embodiments or may be combined in yet other embodiments, further details of which are disclosed with reference to the following description and drawings.

### BRIEF DESCRIPTION OF THE DRAWINGS

FIG. 1 is a diagram of a particular embodiment of an electrocardiogram (ECG) sensor system;

FIG. 2A is a diagram of a particular embodiment of a first side of a patch of the ECG sensor system of FIG. 1;

FIG. 2B is a diagram of a particular embodiment of a second side of a patch of the ECG sensor system of FIG. 1;

FIG. 3 is a diagram of a particular embodiment of a sensor system of an ECG sensor system;

FIG. 4 is a diagram of a particular embodiment of a base system of an ECG sensor system;

FIG. 5 is flow chart of a first particular embodiment of a method performed at an ECG sensor system;

FIG. 6 is a flow chart of a second particular embodiment of a method performed at an ECG sensor system;

FIG. 7 is a flow chart of a third particular embodiment of a method performed at an ECG sensor system;

FIG. 8 is a flow chart of a first particular embodiment of a method performed at an automated troubleshooting system; and

FIG. 9 is a flow chart of a second particular embodiment of a method performed at an automated troubleshooting system.

Illustrative embodiments are described herein. Particular illustrative embodiments of the present disclosure are described below with reference to the drawings. In the description, common elements are designated by common reference numbers throughout the drawings.

### DETAILED DESCRIPTION

A medical device system may include a sensor system that enables a patient to gather and monitor electrocardiogram (ECG) diagnostic information and perform heart beat detection using the ECG diagnostic information at any time with minimal physician and technician assistance. Troubleshooting information provided by the sensor system may facilitate troubleshooting of problems and provide assistance with operation of the sensor system. The troubleshooting information may be determined by using computationally efficient tests that process and analyze the ECG diagnostic information. The troubleshooting information may provide an indication for adjustment and operation of the sensor system to improve accuracy of sensing of the ECG diagnostic information. The ability of the patient to troubleshoot problems with the medical device system may reduce time and cost associated with receiving assistance from a service technician, manufacturer customer support, or a physician to troubleshoot the medical device system. The troubleshooting information can assist a manufacturer or a service technician with correcting problems with the medical device system without performing additional diagnostic tests.

The medical device system may include an automated troubleshooting system that can perform additional troubleshooting and analysis based on the ECG diagnostic information and the troubleshooting information provided by the sensor system. The sensor system may communicate the ECG diagnostic information, heart beat detection information, and the troubleshooting information to the automated troubleshooting system. The automated troubleshooting system may communicate to the sensor system diagnostic information, such as simulated ECG data, that can be used by the sensor system to produce additional troubleshooting information. The ECG diagnostic information and the heart beat detection information may be communicated by the automated troubleshooting system to a remote computing device associated with a healthcare provider, or a manufacturer or distributor of the medical device system, to monitor and perform additional medical diagnosis or troubleshooting.

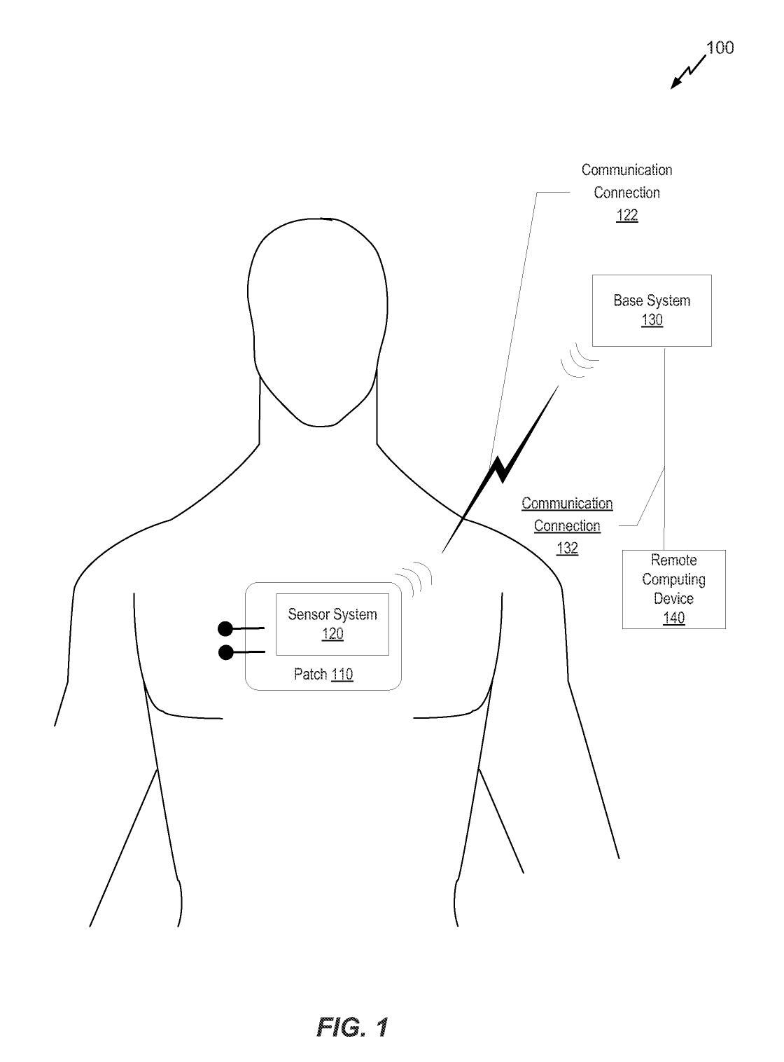

FIG. 1 illustrates a diagram of a particular embodiment of an ECG sensor system 100. The ECG sensor system 100 may include a patch 110, a sensor system 120, a base system 130, and a remote computing device 140. The sensor system 120 may be coupled to the patch 110. The base system 130 may be communicatively coupled to the sensor system 120 via a communication connection 122. The communication connection 122 may include a wired connection, a wireless connection, other data connection, or a combination thereof. The remote computing device 140 may be communicatively coupled to the base system 130 via a communication connection 132.

The patch 110 may be placed on an exterior surface (e.g., skin) of a patient's body. The patch 110 may be configured to sense ECG data produced by the patient's body. For example, the sensed ECG data may include an ECG signal corresponding to an ECG trace produced by the patient's body. The patch 110 may be configured to be coupled to the sensor system 120. When the sensor system 120 is coupled to the patch 110, the patch 110 may provide sensed ECG data to the sensor system 120. The sensor system 120 may be configured to communicate with the base system 130 via the communication connection 122.

The illustration in FIG. 1 depicts the patch 110 positioned on the exterior surface (e.g., skin) of the patient's body in proximity to the patient's chest where an ECG signal may be received from the patient's heart. The patch 110 may be positioned at one or more other locations on the exterior surface of the patient's body that enable an ECG signal to be received from the patient's heart. In a particular embodiment, the patch 110 may be affixed to the exterior surface of the patient's body. The patch 110 may be affixed by an adhesive, a strap, or a combination thereof.

The sensor system 120 may receive the ECG data sensed by the patch 110. In a particular embodiment, the sensor system 120 may be an ECG sensor system. The sensor system 120 may process the sensed ECG data to determine diagnostic information about operation of the patch 110 and the sensor system 120, medical information about the patient, or both. In a particular embodiment, the sensor system 120 may determine whether the sensed ECG data includes information, such as noise, that was introduced by a source other than activity from the patient's heart when the ECG data was sensed. In another particular embodiment, the sensor system 120 may analyze the sensed ECG data to identify potential seizure events and to log the identified potential seizure events that may have occurred during a time period corresponding to when the sensed ECG data was gathered. In another particular embodiment, the sensor system 120 may process the sensed ECG data to produce heart beat detection data. The heart beat detection data may be analyzed by the sensor system 120 to identify potential seizure events. In a particular embodiment, the sensor system 120 may store or log at least a part of the sensed ECG data, the identified potential seizure events, results of processing the sensed ECG data, or a combination thereof. The sensor system 120 may communicate to the base system 130 the sensed ECG data, including information determined based on the ECG data, other data, or a combination thereof. In another embodiment, the ECG data may be provided to the base system 130 to determine whether the ECG data includes noise introduced by a source other than activity from the patient's heart when the ECG data was sensed.

The base system 130 may include an automated troubleshooting system that performs troubleshooting based on received data. The base system 130 may perform troubleshooting for the sensor system 120 based on data received from the sensor system 120. The base system 130 may perform an analysis based on data received from the sensor system 120 to identify problems in operation of the sensor system 120. The base system 130 may output information indicating the identified problems and troubleshooting information associated with the identified problems. The data received from the sensor system 120 may be analyzed by the base system 130 to determine a source of an error identifiable in the sensed ECG data. In some embodiments, at least a portion of the automated troubleshooting system may be part of the sensor system 120. The base system 130 may communicate information to the sensor system 120 to perform further diagnosis of the sensor system 120, to control operation of the sensor system 120, to request additional information from the sensor system 120, or a combination thereof. The base system 130 may communicate information associated with the received data (from the sensor system 120) to the remote computing device 140.

The remote computing device 140 may be a computing device that is located at a location remote from the base system 130. For example, the remote computing device 140 may be at a location associated with a health care provider, such as a hospital. The remote computing device 140 may be configured to communicate with the base system 130 via the communication connection 132. The communication connection 132 may include a wired connection, a wireless connection, other data connection, or a combination thereof. The remote computing device 140 may communicate patient information to the base system 130.

During operation, the patch 110 may sense ECG data when the patch 110 is in contact with the patient's body. The ECG data may be sensed and provided to the sensor system 120 in response to the sensor system 120 being coupled to the patch 110. The sensor system 120 may process the sensed ECG data to determine information based on the sensed ECG data. The sensor system 120 may store in the sensor system 120 and/or communicate to the base system 130 at least a part of the sensed ECG data, information determined based on the sensed ECG data, other data, or a combination thereof. The base system 130 may analyze data received from the sensor system 120 to perform troubleshooting of the patch 110, the sensor system 120, or both. In some embodiments, the sensor system 120 may perform at least a portion of the analysis. The base system 130 may determine whether the sensed ECG data satisfies one or more criteria associated with data errors at the sensor system 120. Based on the determination, the base system 130 may generate an output indicating whether the sensor system 120 has encountered a problem. The base system 130 may communicate the data received from the sensor system 120 to the remote computing device 140. The remote computing device 140 may monitor the patient based on the data received from the base system 130. The output may include information indicating one or more troubleshooting procedures, diagnosis information, or a combination thereof.

Thus, the system described herein reduces effort and involvement by a patient for monitoring a medical condition of the patient. The sensor system enables medical data (e.g., ECG data) to be gathered by a sensor system regularly and periodically without assistance of a physician or a technician. The ability of the sensor system to communicate sensed data to an automated troubleshooting system, which can be further communicated to a health care provider, enables other persons besides the patient to monitor a patient's medical condition. The output provided by the sensor system and the base system provides the patient with information enabling self-diagnostic and self-repair, thereby reducing cost associated with use and maintenance of the sensor system. Self-diagnosis and self-repair of the sensor system may improve performance of the system, such that accuracy of sensed data may be increased.

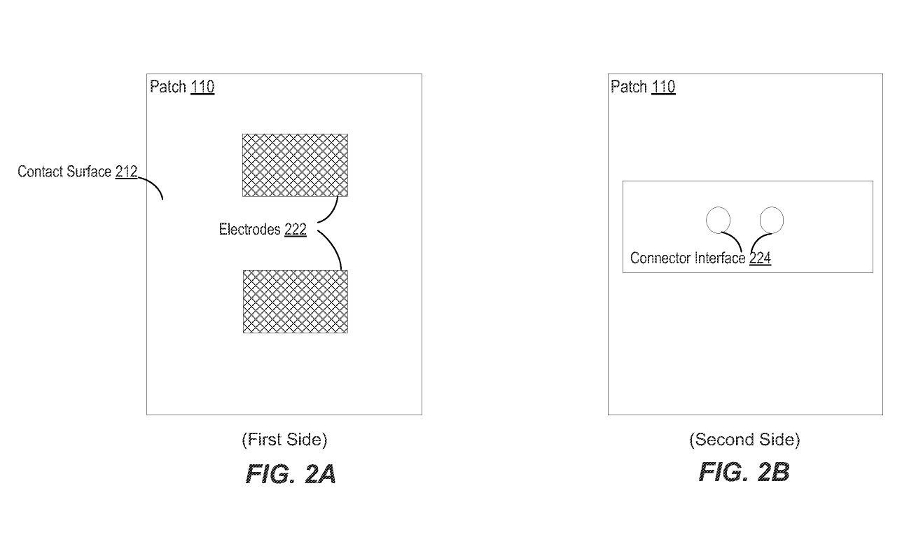

Referring to FIG. 2A, a diagram is illustrated of a particular embodiment of a first side of the patch 110 of the ECG sensor system 100 of FIG. 1. The first side of the patch 110 may include a contact surface 212 and one or more electrodes 222.

The first side of the patch 110 may be configured to contact an exterior surface (e.g., skin) of the patient's body. The contact surface 212 may include an adhesive material that affixes the path 110 to the exterior surface of the patient's body.

In a particular embodiment, the one or more electrodes 222 may each be formed as a mesh grid. The one or more electrodes 222 may be configured to sense ECG data corresponding to an ECG trace produced based on activity of the patient's heart. The one or more electrodes 222 may sense electrical signals corresponding to the ECG data when the patch 110 is positioned on the exterior surface of the patient in proximity to the patient's heart and the one or more electrodes 222 are in electrical contact with the exterior surface.

FIG. 2B is a diagram of a particular embodiment of a second side of the patch 110 of the ECG sensor system 100 of FIG. 1. The second side of the patch 110 may include a connector interface 224. The connector interface 224 may be operatively coupled to the one or more electrodes 222 of the first side of the patch 110. The connector interface 224 may be operatively coupled to one or more interface connectors of a sensor system. For example, the connector interface 224 may be operatively coupled to one or more interface connectors of the sensor system 120. The connector interface 224 may provide the ECG data sensed by the one or more electrodes 222 to the sensor system 120 when the sensor system 120 is operatively coupled to the connector interface 224.

In a particular embodiment, the patch 110 may be disposed of without the sensor system 120. Prior to disposal of the patch 110, the sensor system 120 may be detached from the connector interface 224 of patch 110. The patch may be designed to be disposed of after a period of time (e.g., a week) based on when the patch 110 may have reduced efficacy.

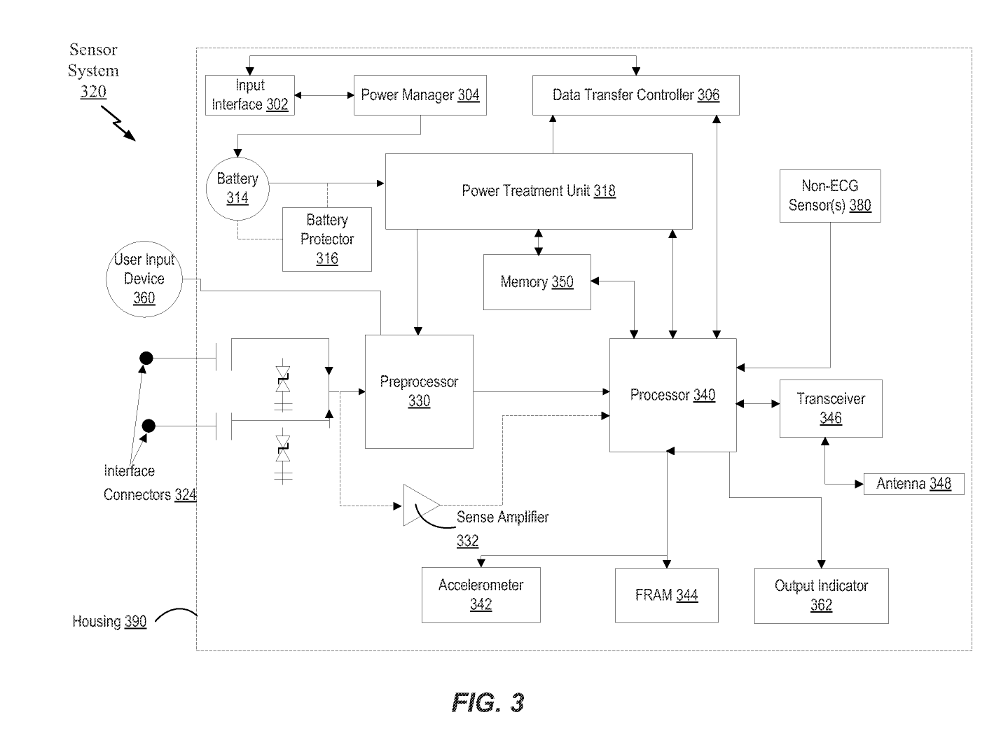

FIG. 3 is a diagram of a particular embodiment of a sensor system 320 of an ECG sensor system. The sensor system 320 may be the sensor system 120 of the ECG sensor system 100 of FIG. 1.

The sensor system 320 may include a preprocessor 330, a processor 340, and a memory 350. The memory 350 may be coupled to the preprocessor 330, to the processor 340, or to both. The memory 350 may include instructions that are executable by a processor (e.g., the preprocessor 330, the processor 340, or both) to operate the sensor system 320. The instructions may further cause the processor to perform one or more of the methods described herein as being performed by a sensor system. The preprocessor 330, the processor 340, or both may include one or more processors. The processor 340 and the preprocessor 330 may be coupled to each other.

The sensor system 320 may include a user input device 360. The user input device 360 may be coupled to the preprocessor 330. The sensor system 320 may include one or more interface connectors 324. The sensor system 320 may include an input interface 302, a power manager 304, a data transfer controller 306, a battery 314, a battery protector 316, a power treatment unit 318, or a combination thereof.

The input interface 302 may be a micro-universal serial bus (USB). The input interface 302 may be coupled to the data transfer controller 306. The data transfer controller may be coupled to the processor 340. The input interface 302 may be coupled to the power manager 304.

The power manager 304 may be coupled to the battery 314 and may control distribution of power to the sensor system 320 by the battery 314. The power manager 304 may be a USB power manager. The battery 314 may be coupled to the battery protector 316. The battery 314 may provide power to a power treatment unit 318. The power treatment unit 318 may control distribution and treatment of the power to the sensor system 320. The power treatment unit 318 may be coupled to the memory 350, the processor 340, the preprocessor 330, and the data transfer controller 306. The power treatment unit 318 may include a buck/boost converter, a boost converter, or a combination thereof.

In a particular embodiment, the sensor system 320 may include a sense amplifier 332. An input of the sense amplifier 332 may be coupled to the one or more interface connectors 324. An output of the sense amplifier 332 may be coupled to the processor 340.

The sensor system 320 may include a transceiver 346 and an antenna 348, coupled to the transceiver 346. The transceiver 346 may be coupled to the processor 340. The sensor system 320 may include a ferroelectric random-access memory (FRAM) 344, an accelerometer 342, one or more non-ECG sensors 380, an output indicator 362, or a combination thereof.

The FRAM 344 may store data and instructions for the processor 340. The FRAM 344 may provide faster memory access. The FRAM 344 may perform access operations faster than access operation performed by the memory 350. The FRAM 344 may operate in the event of a power loss in the sensor system 320. The processor 340 may include the FRAM 344.

The accelerometer 342 may be a 3D accelerometer. The accelerometer 342 may monitor and provide data including activity and movement associated with a patient's body. For example, the accelerometer 342 may provide a measurement associated with movement of the patient's chest such as associated with chest compressions. In another example, the accelerometer 342 may provide a measurement associated with movement of the patient's body. The accelerometer 342 may provide the data, including activity and movement associated with the patient's body, to the processor 340.

The one or more non-ECG sensors 380 may be configured to sense non-ECG data. The non-ECG data may be stored in the memory 350. The non-ECG data may include non-heart beat data, which does not include heart beat information. The non-ECG data may include a non-ECG signal that may correspond to one or more types of noise detectable via the non-ECG sensors 380. For example, the non-ECG sensors 380 may sense electrical activity generated by muscle activity or movement within the patient's body. In this example, the non-ECG data may include an EMG signal which may be indicative of time and intensity of muscle activity (e.g., muscle contraction). In another example, the non-ECG sensors 380 may include a sensor that detects power-line noise (e.g., EMI). The non-ECG data may include one or more measures of EMI such as signal-to-noise ratio (SNR) and signal-to-interference (SIR) ratio.

The output indicator 362 may provide the patient with information associated with the sensor system 320. The information associated with the sensor system 320 may include information associated with a status of the sensor system 320, performance of the sensor system 320, operation of the sensor system 320, troubleshooting information, or a combination thereof. The information provided by the output indicator 362 may indicate one or more predetermined error codes that are selected to facilitate further troubleshooting. The information provided by the output indicator 362 may be stored in the memory 350.

The output indicator 362 may be located at least partially on an exterior surface of the sensor system 320 where the output indicator 362 can provide an output to the patient indicating information associated with the sensor system 320. The output indicator 362 may include one or more light-emitting diodes (LEDs) to provide the indication. In a particular embodiment, the output indicator 362 may indicate to the user that the sensor system 320 should be adjusted. For example, the output indicator 362 may indicate that a connection between the sensor system 320 and the patch 110 at the connector interface 224 should be checked.

The sensor system 320 may be entirely or may be at least partially enclosed by a housing 390. In a particular embodiment, the housing 390 may at least partially enclose the one or more interface connectors 324, the preprocessor 330, the processor 340, and the transceiver 346. The housing 390 may provide water-resistant protection for the one or more interface connectors 324, the preprocessor 330, the processor 340, and the transceiver 346.

The one or more interface connectors 324 may at least partially extend outside of the housing 390. The one or more interface connectors 324 may be operatively coupled to the connector interface 224 of FIG. 2B of the patch 110. The one or more interface connectors 324 may be operatively coupled to the one or more electrodes 212 of FIG. 2B of the patch 110 via the connector interface 224. The one or more interface connectors 324 may receive ECG data, via the connector interface 224, sensed by the one or more electrodes 222. The sensed ECG data, which may include an ECG signal, received by the one or more interface connectors 324 may be provided to the preprocessor 330. The one or more interface connectors 324 may be operatively coupled to a base system of an ECG sensor system, where the base system includes a connector interface that is configured to receive one or more interface connectors. For example, the one or more interface connectors may be operatively coupled to the base system 130 of the ECG sensor system 100.

The preprocessor 330 may perform one or more functions in response to receiving the sensed ECG data from the one or more interface connectors 324. In a particular embodiment, the preprocessor 330 may be a sensing application-specific integrated circuit (ASIC). The preprocessor 330 may process the sensed ECG data to produce processed ECG data. The processed ECG data may include heart beat information determined based on the ECG data. For example, the preprocessor 330 may perform heart beat detection, such as R-wave detection, based on the ECG signal included in the sensed ECG data to produce the heart beat detection information. The heart beat detection information may include heart rate data. The heart rate data can be used as to identify occurrences of potential seizure events.

In a particular embodiment, the preprocessor 330 may amplify the ECG signal included in the sensed ECG data. The preprocessor 330 may amplify the ECG signal based on a determination that the ECG signal does not satisfy one or more criteria that define an acceptable ECG signal. For example, the one or more criteria may define a range based on a component of the ECG signal. When the ECG signal is not within range associated with the particular component of the ECG signal, one or more errors may be attributed to the ECG signal. The ECG signal may be amplified to accommodate for the one or more errors. The preprocessor 330 may be configured to output the sensed ECG data, the amplified ECG signal, the processed ECG data, or a combination thereof.

The sense amplifier 332 may be configured to receive the ECG data from the one or more interface connectors 324. The sense amplifier 332 may include an ECG sense amplifier. The sense amplifier 332 may be configured to operate in parallel with the preprocessor 330. The sense amplifier 332 may amplify the sensed ECG signal. The sense amplifier 332 may be disabled in particular operational modes of the sensor system 320. In an operational mode in which the sense amplifier 332 is enabled, the sensor system 320 may consume additional power to operate the sense amplifier 332.

The sensor system 320 may function in one or more operational modes. The one or more operational mode may each be associated with a different amount of power usage. In a particular embodiment, the sensor system 320 may operate in a first (e.g., default) operational mode in which the preprocessor 330 is enabled and the sense amplifier 332 is disabled. Upon a determination by the preprocessor 330, the processor 340, or another device (e.g., the base system of an ECG sensor system) that the ECG signal has one or more errors, the sensor system 320 may be configured to switch operational modes to a second operational mode. In a particular embodiment, the sensor system 320 may disable the preprocessor 330 in the second operational mode and may enable the sense amplifier 332. The sense amplifier 332 may amplify the sensed ECG signal and the processor 340 may perform heart beat detection. An amount of power consumed from the battery 314 may vary based on the operational mode. In a particular embodiment, the sensor system 320 may consume more power from the battery 314 to operate the sense amplifier 332. Thus, the first operational mode may be a lower power usage mode than the second operational mode.

The processor 340 may be configured to receive output from the preprocessor 330 (e.g., in the first operational mode). The processor 340 may be configured to receive an ECG signal output from the sense amplifier 332 (e.g., in the second operational mode). In a particular embodiment, the processor 340 is a microprocessor (e.g., 16-bit microcontroller). The processor 340 may analyze the output received from the preprocessor 330 to detect one or more potential seizure events. For example, the processor 340 may analyze the heart beat detection information to detect the one or more seizure events. The processor 340 may store a log of the detected potential seizure events in the memory 350. In another particular embodiment in which the sensor system 320 is operating in a particular operational mode (e.g., the second operational mode) in which the preprocessor 330 is disabled, the processor 340 may perform heart beat detection in lieu of the preprocessor 330. In this embodiment, the processor 340 may also perform seizure event detection to detect one or more potential seizure events.

The processor 340 may be operable to process ECG data to identify one or more types of noise that may be detectable in the ECG data. For example, each of the one or more types of noise may be detectable as a component of the ECG signal in the ECG data. Heart beat detection performed based on the ECG signal may be impacted based on the one or more types of noise present in the ECG signal. As a result, heart beat detection may impact identification of seizure events. The one or more types of noise may be produced by a source other than a heart beat related source. For example, the one or more types of noise may include power-line noise, baseline wander, electromyography (EMG) noise (e.g., muscle activity), electronics-related noise, other types of noise associated with one or particular artifacts, or a combination thereof. The processor 340 may be able to detect other types of noise within the sensed ECG data. The processor 340 may determine one or more noise values associated with the sensed ECG data, where each noise value is indicative of a measurement of a component of the sensed ECG data that is potentially related to a non-heart beat source. In a particular embodiment, the processor 340 may periodically, based on a schedule (e.g., hourly, daily, weekly, and monthly), process the sensed ECG data to determine the one or more noise values. The schedule may be stored in the memory 350. The processor 340 may store the one or more noise values in the memory 350. In some embodiments, a processor of a base system associated with the sensor system 320 may be configured to determine at least a portion of the one or more noise values associated with the sensed ECG data.

In a particular embodiment, the processor 340 may generate an estimate of power-line noise in the sensed ECG data. Power-line noise may be caused by or associated with radiation of energy dissipated by power-lines. For example, the power-line noise may be associated with electromagnetic interference (EMI), which may be caused by radiation of energy produced by power-lines. The processor 340 may generate an estimate of baseline wander in the sensed ECG data. Baseline wander may correspond to low frequency noise in the sensed ECG signal. An estimate of the baseline wander may provide an indication of an amount of baseline wander present in the sensed ECG signal. The processor 340 may generate an estimate of EMG noise in the sensed ECG data. EMG noise may correspond to movement in patient, such as muscle movement/activity within the patient's body.

The processor 340 may generate an estimate of electronics noise of the ECG sensor system in the sensed ECG data. Electronics noise may be associated with noise introduced in the sensed ECG data by operation of one or more electric components. The one or more electronic components may be included in the sensor system 320. For example, the electronics noise may be associated with a write operation performed by the processor 340 using the memory 350, the FRAM 344, or both. In another example, the electronics noise may be associated with operation/activity of the transceiver 346, the antenna 348, or both. In another example, the electronics noise may be associated with interference caused by power transmission by the battery 314.

In a particular embodiment, estimating electronics noise may include applying a wavelet filter to the sensed ECG data to detect a spike, or a pattern in a sample portion of the sensed ECG data. The spike in the sample portion of the sensed ECG data may correspond to noise introduced into the sensed ECG data. In another particular embodiment, estimating the electronics noise may include applying a filter that is adapted to pass pre-determined electronic signal artifacts within a sample portion of the sensed ECG data. The predetermined electronic signal artifacts may correspond to signal artifacts that are known to be generated by components of the ECG sensor system. In another particular embodiment, estimating the electronics noise may include comparing a sample portion of the sensed ECG data to a log of the sensor system 320 activity to identify signal artifacts generated by the ECG sensor system.

The processor 340 may be configured to maintain a log of system activity within the sensor system 320. The log of system activity may include communication activity of the sensor system 320. The communication activity may include activation and deactivation activity performed by the transceiver 346. The log of system activity may include memory activity including operation of the memory 350, the FRAM 344, or both. The memory activity may include memory read and write operations.

The processor 340 may store noise information associated with the one or more noise values, which may include estimates of the one or more noise values, in the memory 350. Based on the noise information stored in the memory 350, a patient or a technician to troubleshoot operation and performance of the sensor system 320 to improve diagnosis of problems with the sensor system 320. The noise information may provide an indication as to the quality of the sensed ECG data such that a determination can be made as to whether the sensed ECG data is accurate and reliable. For example, a presence of one or more types of noise may indicate that the sensed ECG data is not accurate or should not be used to detect heart beat data or to determine seizures events.

The sensor system 320 may be configured to adjust one or more components of the sensor system 320 based on information associated with the one or more noise values determined by the processor 340. For example, the sensor system 320 may be configured to adjust one or more components of the sensor system 320 to reduce one or more noise values determined based on the sensed ECG data. The one or more components may be adjusted by being activated, deactivated or adjusted to an operational mode that may cause a reduction in at least one noise value. The operational mode may include a low-power mode such that the one or more components operate using a reduced amount of power.

The transceiver 346 may configured to communicate with one or more external devices. The one or more external devices may include the base system 130 of FIG. 1. The transceiver 346 may perform transmission via the antenna 348. The transceiver 346 may include a transmitter to transmit communications signals. and a receiver to receive communication signals. The sensor system 320 may use the transceiver 346 to communicate with an external device via a communication connection. The communication connection may include a wireless connection, another data connection, or both. The communication connection may facilitate data communication according to one or more of wireless mobile data communication compliant standards including code division multiple access (CDMA), time division multiple access (TDMA), frequency division multiple access (FDMA), orthogonal frequency division multiple access (OFDMA), single-carrier frequency division multiple access (SC-FDMA), a global system for mobile communications (GSM), enhanced data rates for GSM evolution (EDGE), evolved EDGE, Universal Mobile Telecommunications System (UMTS), Worldwide Interoperability for Microwave Access (Wi-Max), general packet radio service (GPRS), 3rd generation partnership project (3GPP), 3GPP2, 4th generation (4G), long term evolution (LTE), 4G-LTE, high speed packet access (HSPA), HSPA+, Institute of Electrical and Electronics Engineers (IEEE) 802.11x, or a combination thereof.

The transceiver 346 may be operable to send data to one or more external devices. The transceiver 346 may be operable to send data stored in the memory 350, the FRAM 344, or both, to the one or more external devices. For example, the transceiver 346 may send the one or more noise values to the base system 130. In another example, the transceiver 346 may send non-ECG data, sensed from the one or more non-ECG sensors 380, to the base system 130. The transceiver 346 may be operable to send seizure event data, determined by the processor 340, to the base system 130.

The transceiver 346 may be operable to receive data from the one or more external devices. For example, the transceiver 346 may receive data from an automated troubleshooting system (e.g., the base system 130). In a particular embodiment, the sensor system 320 may receive output from the automated troubleshooting system that causes the ECG sensor system 320 to switch from one operational mode to another operational mode. The output from the automated troubleshooting system may be based on analysis of data communicated from the sensor system 320 to the automated troubleshooting system. In another particular embodiment, the sensor system 320 may receive a simulated ECG signal from the automated troubleshooting system. The simulated ECG signal may be a “clean” ECG signal such that the simulated ECG signal does not include components from non-heart beat sources (e.g., one or more types of noise). The simulated ECG signal may be provided to the sensor system 320 during a test mode of operation. In some embodiments, at least a portion of the automated troubleshooting system may be part of the sensor system 320.

The processor 340 may be configured to process data received from the automated troubleshooting system. In a particular embodiment, the processor 340 may process the simulated ECG signal to determine one or more second noise values. The one or more second noise values may be useful in determining whether the sensor system 320 is producing one or more types of noise that cause a noise component to be added to an ECG signal processed by the sensor system 320. By determining one or more second noise values based on a “clean” ECG signal, the one or more second noise values may be indicative of whether the sensor system 320 is a source of one or more types of noise. The sensor system 320 may send the one or more second noise values to the automated troubleshooting system to further troubleshoot the sensor system 320.

In a particular embodiment, the output indicator 362 may indicate that the patch 110 should be re-positioned or placed at another location on the exterior surface of the user. A determination that the patch 110 should be re-positioned or placed at another location on the patient's body may be based on a determination of the presence of one or more noise values. In a particular embodiment, the output indicator 362 may indicate that the patch 110 should be replaced. In a particular embodiment, the output indicator 362 may provide information indicating one or more sources affecting the sensed ECG data. For example, the information may indicate that excessive movement of the sensor system 320 is affecting the sensed ECG data. In another particular embodiment, the output indicator 362 may provide an indication that the sensor system 320 is not operating properly based on in part on a determination that one or more noise values are present. The output indicator may indicate that the sensor system 320 should be operated in a high power mode. The output indicator 362 may indicate that one or more components of the sensor system 320 should be replaced. The output indicator 362 may indicate that the sensor system 320 should be connected to the base system 130 to obtain additional information regarding a state of the sensor system 320.

The user input device 360 may enable the patient to provide input to the sensor system 320. The input may be used to control operation of the sensor system 320. For example, the user input device 360 may be configured to cause the processor 340 to process the sensed ECG data in response to user input via the user input device 360. In response to a user request via the user input device 360, the processor 340 may determine the one or more noise values associated with the ECG signal. In another example, the user input device 360 may control selection of one or more operational modes of the sensor system 320.

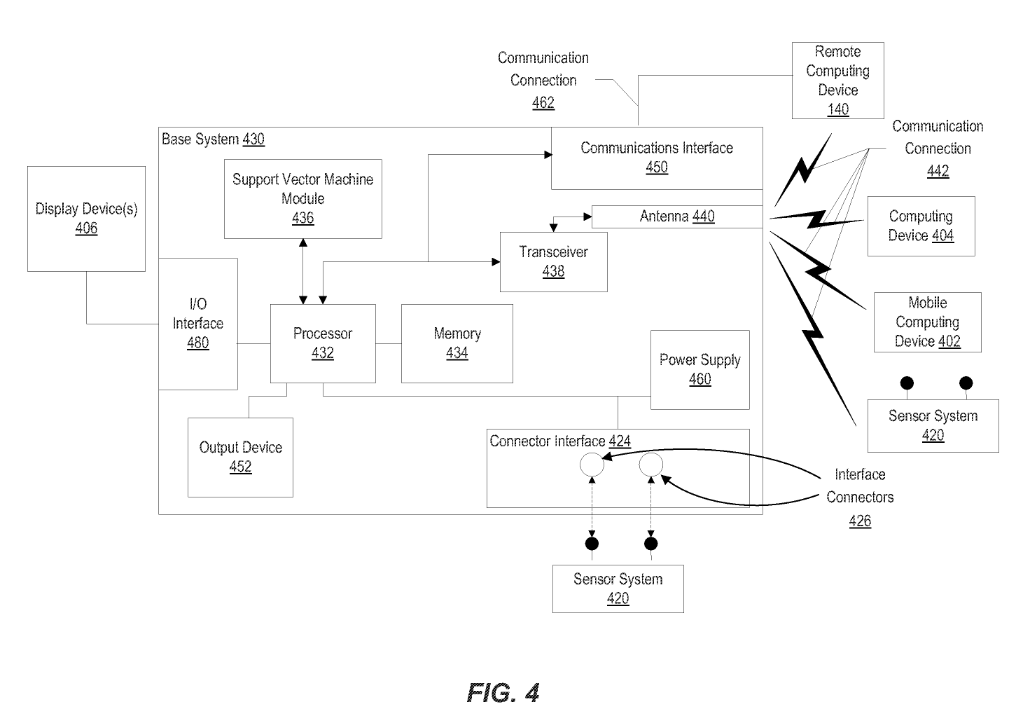

FIG. 4 is a diagram of a particular embodiment of a base system 430 of an ECG sensor system. The base system 430 may be the base system 130 of the ECG sensor system 100 of FIG. 1. In a particular embodiment, the base system 430 may include an automated troubleshooting system.

The base system 430 may include a processor 432 and a memory 434. The memory 434 may be coupled to the processor 432. The processor 432 may include one or more processors. The instructions may be executable by the processor 432 to cause the processor 432 to perform one or more of the functions of an automated troubleshooting system, the base system 130, the base system 430, or a combination thereof.

The base system 430 may include a transceiver 438. The transceiver 438 may be coupled to the processor 432. The base system 430 may include an antenna 440 that is coupled to the transceiver 438. The base system 430 may include a communications interface 450, a connector interface 424, or both.

The base system 430 may include a power supply 460, a support vector machine (SVM) 436, an input/output (I/O) interface 480, an output device 452, or a combination thereof. The power supply 460 may be coupled to the connector interface 424, the processor 432, or both. The processor 432 may be coupled to the I/O interface 480. The I/O interface 480 may be coupled to one or more display devices 406. The output device 452 may be coupled to the processor 432.

The base system 430 may be configured to communicate, via a communication connection 442, 462, with one or more devices, one or more systems, or both, that are located externally from the base system 430. The communication connection 462 may include a wired connection, a wireless connection, another data connection, or a combination thereof. The communication connection 442, 462 may operate based on one or more of wireless mobile data communication compliant standards including CDMA, TDMA, FDMA, OFDMA, SC-FDMA, GSM, EDGE, evolved EDGE, UMTS, Wi-Max, GPRS, 3GPP, 3GPP2, 4G, LTE, 4G-LTE, HSPA, HSPA+, IEEE 802.11x, or a combination thereof. In a particular embodiment, the communications interface 450 may establish and manage the communication connection 462. The communication connection 442 may be established and managed by the transceiver 438 that sends and receives communications signals via the antenna 440.

In a particular embodiment, the one or more devices may include an external computing device (such as the remote computing device 140) located at a health care provider. In a particular embodiment, the one or more devices, the one or more systems, or both may include a mobile computing device 402, a computing device 404, a sensor system 420, or a combination thereof. The sensor system 420 may be the sensor system 120 of FIG. 1 or the sensor system 320 of FIG. 3.

The connector interface 424 may be configured to receive one or more interface connectors of the sensor system 420. The connector interface 424 may include one or more interface connectors 426, which may be configured to physical couple to one or more interface connectors of the sensor system 420. In a particular embodiment, the base system 430 may be configured to provide power, communicate data, or both to the sensor system 420, when the sensor system 420 is coupled to the connector interface 424. For example, the one or more interface connectors 324 of sensor system 320 of FIG. 3 may be coupled to the connector interface 424 to receive power, data, or both from the base system 430. The sensor system 420 may be provided power from the power supply 460. The data communicated to the sensor system 420 via the connector interface 424 may include data that resides within the processor 432, the memory 434, or both. The data may be produced as a result of operation of the base system 430. The connector interface 424 may be configured to provide an electrical output representing one or more ECG signals to one or more corresponding interface connectors of the sensor system 420.

The base system 430 may provide automated troubleshooting information based on data received from the one or more devices, the one or more systems, or both. The base system 430 may perform an analysis based on the data received to determine troubleshooting information. For example, the base system 430 may perform an analysis based on data received from the sensor system 420 to identify problems in operation of the sensor system 420 and to provide an indication of the identified problems. In a particular embodiment, the data received from the sensor system 420 may include one or more noise values determined by the sensor system 420. In a particular embodiment, the base system 430 may perform an analysis on the one or more noise values to identify a potential source of a particular component of the sensed ECG data that is potentially related to a particular non-heart beat source based on the analysis. The one or more noise values may each be indicative of a measurement of a component of ECG data. The processor 432 may determine, based on the one or more noise values, whether the ECG data corresponding to sensed ECG signal satisfies one or more criteria. The memory 434 may include the one or more criteria that may be defined by one or more values that identify a type of noise attributed to the particular non-heart beat source. In some embodiments, the base system 430 may be configured to determine at least a portion of the one or more noise values from the sensed ECG data.

In a particular embodiment, the one or more criteria may include one or more predetermined threshold noise values that identify a type of noise attributed to a particular non-heart beat source. In an illustrative example, the one or more values of the data received from the sensor system 420 may be compared to one or more of the predetermined threshold noise values corresponding to a type of noise to determine whether the one or more values satisfies one or more of the predetermined threshold values. A type of noise, attributable to a particular non-heart beat source, may be present as a component within the data received from the sensor based on determining that the one or more noise values of the data satisfies one or more of the predetermined threshold noise values.

The processor 432 may analyze the received data to determine a source of an error corresponding to the sensed ECG data. In a particular embodiment, the base system 430 may use the SVM 436 to compare the one or more noise values to the one or more criteria. The SVM 436 may determine a source of the error based on whether the one or more criteria are satisfied.

The SVM 436 may be a part of the processor 432, or may be a separate component of the base system 430 coupled to the processor 432. As a separate component, the SVM 436 may include one or more processors and memory. The SVM 436 may analyze data and recognize patterns in the sensed ECG data. For example, the SVM 436 may use a supervised learning model associated with a classification algorithm to analyze the sensed ECG data to determine whether the sensed ECG data satisfies criteria that define one or more types of noise may be present in an ECG signal. In a particular embodiment, the SVM 436 may apply a particular classification algorithm to determine whether the one or more noise values satisfy the one or more criteria. The one or more criteria may be stored in memory associated with the SVM 436 or may be provided to the SVM 436 by the processor 432. The one or more criteria may correspond to one or more threshold value ranges defining one or more types of noise. The classification algorithm may identify the one or more types of noise based on the supervised learning model that is defined based on the one or more criteria. To identify the one or more types of noise, the classification algorithm may determine whether the one or more noise values are within an acceptable range corresponding to each of the one or more threshold ranges defining the one or more types of noise.

The base system 430 may communicate to the remote computing device 140 information associated with data received from the sensor system 420. The received data may include the sensed ECG data obtained from the patient. The received data may include seizure event data determined by the sensor system 320 based the sensed ECG data. The base system 430 may also send other data received from the sensor system 420.

The base system 430 may communicate information to the sensor system 420 to perform further diagnosis of the sensor system 420. For example, the base system 430 may send a simulated ECG signal to the sensor system 420. To illustrate, the simulated ECG signal may be retrieved from the memory 434 that stores a representation of the simulated ECG signal. The simulated ECG signal may be sent to the sensor system 420 to diagnose a problem at the sensor system, which may be related to one or more types of noise occurring at the sensor system 420. The connector interface 424 may be configured to provide an electrical output corresponding to the simulated ECG signal to the one or more corresponding interface connectors of the sensor system during a test mode of operation. One type of simulated ECG signal may be a “clean” ECG signal. The clean ECG signal may be based on previously detected heart rate information for a particular patient. The clean ECG signal may not include one or more components that are related to a non-heart beat source such as a type of noise. Another type of simulated ECG signal may be based on known heart rate information for a particular patient, but may include one or more known artifacts that are associated with a component related to a non-heart beat source. The sensor system 420 may receive the clean ECG signal while coupled to the connector interface 424. The clean ECG signal received via the electrical output from the connector interface 424. The clean ECG signal may be similar in format the sensed ECG signal received from the patch 110. Therefore, the base system 430 may detect errors introduced by the sensor system 420 based on output provided from the sensor system 420.

The base system 430 may receive information from the sensor system 420 to perform further diagnosis of the sensor system 420. In a particular embodiment, the base system 430 may receive one or more second noise values from the sensor system 420, where the one or more second noise values are determined based on the simulated ECG signal. The one or more second noise values may correspond to one or more types of noise that may be detectable as a component of the simulated ECG signal. The one or more types of noise may include power-line noise, baseline wander, EMG noise, electronics-related noise, other types of noise associated with one or particular artifacts, or a combination thereof. The processor 432 may determine one or more suspected or potential causes of the one or more second noise values. The processor 432 may determine whether the one or more second noise values are indicative of noise attributable to a particular non-heart beat source. The processor 432 may process the one or more second noise values. For example, the processor 432 may compare the one or more second noise values to one or more criteria that may be associated with one or more measures associated with a non-heart beat source. The base system 430 may use the SVM 436 to compare the one or more second noise values to the one or more criteria to make a determination as to the suspected causes of noise.

The base system 430 may determine an output to generate based on analysis of data received from the sensor system 420. Based on the output determined by the base system 430, the base system 430 may output information that may be used to troubleshoot problems with the sensor system 420. The output information may include recommended troubleshooting procedures. The recommended troubleshooting procedures may be determined from data stored the memory 434. The output information may include one or more error codes selected to facilitate further troubleshooting. The base system 430 may include other diagnostic and troubleshooting information, which may be stored in the memory 434, to assist in troubleshooting of the sensor system 420. The output may further include event history that is descriptive of operations performed at the base system 430.

The base system 430 may communicate the output, determined based on analysis of the data received from the sensor system 420, to one or more devices (e.g., the computing device 404, the mobile computing device 402, and the sensor system 420). The base system 430 may provide the output indicating troubleshooting information, via the output device 452, to a patient. The base system 430 may communicate the output to the one or more display devices 406 via the I/O interface 480.

The base system 430 may receive input via a user interface of the one or more display devices 406. The input may control operation of the base system 430. For example, the input from the one or more display devices 406 may indicate confirmation/denial of actions to be performed by the base system. In another example, the input may include feedback by the patient that indicates whether the troubleshooting procedures have resolved a problem with the sensor system 420.

The base system 430 may control operation of the sensor system 420. In a particular embodiment, the base system 430 may send one or more signals to the sensor system 420 to cause the sensor system 420 to control selection of an operational mode. A signal may be sent in response to determining that the one or more noise values indicates that the ECG data sensed by the sensor system 420 does not satisfy the one or more criteria. In a particular embodiment, the one or more criteria may be defined by one or more threshold level values that correspond to one or more types of noise. In one example, the base system 430 may determine that the one or noise values does not satisfy the one or more criteria indicating that the preprocessor 330 of the sensor system 320 is producing noise on the sensed ECG data. The base system 430 may send a signal to the sensor system 420 to select an operational mode of the sensor system 420 to disable the preprocessor 330 and to enable the sense amplifier 332.

The base system 430 may be a stand-a-lone device or may be distributed across multiple devices. Portions of the functionality of the base system 430 described in this disclosure, including the automated troubleshooting system, may be implemented or performed in the sensor system 320. Portions of the functionality of the sensor system 320 described in this disclosure may be implemented or performed in the base system 430. The base system 430 may be a custom built device, a personal computer, or a mobile computing device, such as a laptop computer, a personal digital assistant (PDA), a smart phone, a tablet, or any other type of mobile or hand held computing device. The personal computer or mobile computing device may serve as at least a portion the base system 430 and may include software, such as a software application (e.g., an app) to perform one or more of the functions described in this disclosure.

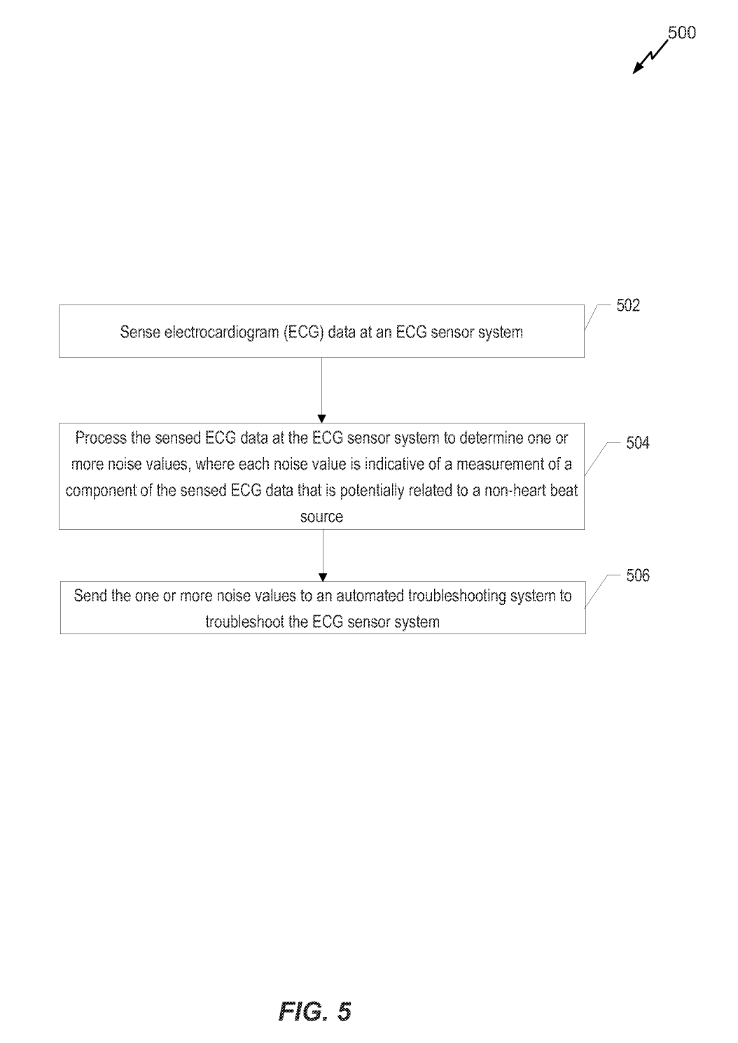

FIG. 5 is flow chart of a first particular embodiment of a method 500 performed at an ECG sensor system. For example, the method 500 may be performed by an ECG sensor system, such as the sensor system 120 of FIG. 1 or the sensor system 320 of FIG. 3.

At 502, the method 500 may include sensing ECG data at an ECG sensor system. For example, the sensor system 120 of FIG. 1 may sense the ECG data. In another example, the sensor system 320 of FIG. 3 may sense the ECG data.

At 504, the method 500 may include processing the sensed ECG data at the ECG sensor system to determine one or more noise values, where each noise value is indicative of a measurement of a component of the sensed ECG data that is potentially related to a non-heart beat source. For example, the sensor system 320 of FIG. 3 may process the sensed ECG data to determine the one or more noise values. To illustrate, the processor 340 of the sensor system 320 may receive the sensed ECG data from the preprocessor 330, and the processor 340 may process the sensed ECG data to determine the one or more noise values. In some embodiments, the ECG data may be sent from the sensor system 320 to the base system 430 and the base system 430 may be configured to determine at least a portion of the one or more noise values.

The one or more noise values may correspond to one or more types of noise that may be detectable in the sensed ECG data. The one or more types of noise may correspond to a component of the sensed ECG data that is associated with or subjects the sensed ECG to errors when the sensed ECG data is processed to determine heart beat information and identify seizure events. The errors may cause incorrect or inaccurate results to be produced. For example, the one or more types of noise may include power-line noise, baseline wander, EMG noise, electronics-related noise, other types of noise associated with one or particular artifacts, or a combination thereof.

In a particular embodiment, processing the sensed ECG data to determine the one or more noise values may include generating an estimate of power-line noise in the sensed ECG data. In a particular embodiment, an estimate of the power-line noise may be generated based on computing a signal-to-noise ratio (SNR) value based on the sensed ECG signal. In estimating the SNR value, a sample frame of the sensed ECG data may be filtered based on a particular power-line frequency to identify a particular portion of the sensed ECG data of the sample frame that corresponds to the particular power-line frequency. The SNR value may be indicative of a presence of power-line noise and a strength of the sensed ECG signal at the particular frequency range associated with power-line frequency.

In a particular embodiment, the SNR value may be computed according to the following equation:

SNR_{BP60}=Amplitude(p−p)_{QRS}/RMS_{BP60}; (Eqn 1)

where, Amplitude (p−p)_{QRS} is the amplitude of the sensed ECG signal associated with a QRS complex; and
where, RMS_{BP60} may be calculated based band-pass filtered data for the particular frequency range (e.g., 58 Hz-62 Hz) outside a PQRS complex.

To compute the power-line noise, a root mean square (“RMS”) value (e.g., RMS_{BP60}) may be calculated. The RMS value may be determined based on measuring amplitude of the sensed ECG signal when a band-pass filter defined by a particular frequency range is applied to a sample frame of the sensed ECG data. An amplitude of the sensed ECG signal (e.g., Amplitude (p−p)_{QRS}) associated with a QRS complex of the sensed ECG signal may be determined. Following Eqn 1, a SNR value (e.g., SNR_{BP60}) may be may be calculated according to a ratio of the amplitude of the sensed ECG signal that is band-pass filtered to the RMS value. The SNR value corresponds to an amount of power-line noise that is present in the sensed ECG signal. A greater SNR value corresponds to a larger amount of power-line noise.

In another particular embodiment, processing the sensed ECG data to determine the one or more noise values may include generating an estimate of the baseline wander in the sensed ECG data. The estimate of the baseline wander may be generated by computing a baseline wander value based on the sensed ECG signal. Estimating the baseline wander value may include measuring amplitude of a sample frame of the sensed ECG data (e.g., sensed ECG signal) after a low-pass filter is applied. The baseline wander value may indicate a presence of baseline wander and a strength of the baseline wander in the sensed ECG signal. In a particular embodiment, the baseline wander value may be computed according to the following equation:

Baseline Wander=ABS(Mean_{LPF}); (Eqn 2);

where low-pass filter (LPF) is defined as a low-pass filter at 1 Hz;
where Mean_{LPF} is the mean value of a measure of amplitude of the sensed ECG signal applied with a low-pass filter; and
where ABS is the absolute value of the Mean_{LPF}.

To compute the baseline wander, a mean value (e.g., the Mean_{LPF}) may be calculated based on a measure of an amplitude of a sample frame of the sensed ECG data after a low-pass filter is applied. To apply the low-pass filter, a band-pass filter may be applied based on a particular frequency that defines the low-pass filter. The particular frequency may correspond to a threshold frequency delineating a presence of a baseline wander. In a particular embodiment, the low-pass filter may be defined by a frequency of 1 Hz, below which baseline wander may be present in the sensed ECG signal. An estimate of the baseline wander may be determined based on an absolute value, the ABS (Mean_{LPF}), which may be calculated based on the mean value of amplitude of the sensed ECG data after the low-pass filter is applied. The baseline wander value may indicate a strength of baseline wander in the sensed ECG signal below the frequency of the low-pass filter (e.g., 1 Hz).

In another particular embodiment, processing the sensed ECG data to determine the one or more noise values may include generating an estimate of EMG noise in the sensed ECG data. Determining the estimate of the EMG noise may be generated by computing an EMG noise value based on the sensed ECG data (e.g., the sensed ECG signal). Estimating the EMG noise may include measuring amplitude of a sample frame of the sensed ECG data after a high-pass filter is applied. The estimate of the EMG noise may indicate presence of EMG noise and a strength of the EMG noise in the sensed ECG signal above the frequency of the high-pass filter. In a particular embodiment, EMG noise may be determined using the following equation:

EMG Noise=ABS(Mean_{HPF}); (Eqn 3)

where high-pass filter (HPF) is defined as a high-pass filter at 25 Hz;
where Mean_{HPF} is the mean value of a measure of amplitude of the sensed ECG signal applied with the HPF; and
where ABS is the absolute value of the Mean_{HPF}.

To compute the EMG noise, a mean value (e.g., the Mean_{HPF}) may be calculated based on a measure of amplitude of a sample frame of the sensed ECG data after a high-pass filter is applied. To apply the high-pass filter, a band-pass filter may be applied based on a particular frequency that defines the high-pass filter. The frequency may correspond to a threshold frequency that is used to delineate whether EMG noise is present. In a particular embodiment, the high-pass filter may be defined by a frequency of 25 Hz, at or above which EMG noise may be determined to be present in the sensed ECG signal. An estimate of the EMG noise may be based on an absolute value, the ABS (Mean_{HPF}), which may be calculated based on the mean value of the amplitude of the sensed ECG data after the high-pass filter is applied. The estimate of the EMG noise may indicate a strength of EMG noise in the sensed ECG signal above the frequency of the high-pass filter.

In another particular embodiment, determining the estimate of the EMG noise may include receiving movement data from an accelerometer and generating an estimate of the EMG noise by comparing the movement data to the sensed ECG data. For example, the sensor system 320 of FIG. 3 may receive movement data from the accelerometer 342. The sensor system 320 may compare one or more components of the sensed ECG data to the movement data to determine whether the movement data indicates a pattern that corresponds to a presence of EMG noise. The estimate of the EMG noise may be based on a measurement of the pattern identified within the movement data.

In another particular embodiment, processing the sensed ECG data to determine the one or more noise values may include generating an estimate of electronics noise in the sensed ECG data. Electronics noise may be associated with noise introduced in the sensed ECG data by operation of one or more electric components. In a particular embodiment, generating an estimate of the electronics noise may include applying a wavelet filter to the sensed ECG data to detect a spike, or a pattern in a sample portion of the sensed ECG data. The spike in the sample portion of the sensed ECG data may be noise introduced into the sensed ECG data. The estimate of the electronics noise may be based on a measurement of amplitude of the detected spike.

In another particular embodiment, generating an estimate of the electronics noise of the ECG sensor system may include applying a filter that is adapted to pass predetermined electronic signal artifacts within a sample portion of the sensed ECG data. The estimate of the electronics noise may be based on a measure of an amplitude associated with one or more of the predetermined electronic signal artifacts. The predetermined electronic signal artifacts may correspond to signal artifacts that are known to be generated by the ECG sensor system. For example, the signal artifacts may correspond to activity of a transmitter of the transceiver 346 of FIG. 3.

In another particular embodiment, generating an estimate of the electronics noise of the ECG sensor system may include comparing a sample portion of the sensed ECG data to a log of ECG sensor system activity to identify signal artifacts generated by the ECG sensor system. The estimate of the electronics noise may be based on a measurement of amplitude of the sample portion of the sensed ECG data in corresponding to the signal artifacts that are identified. The log of ECG sensor system activity that is identified based on a comparison of the sample portion of the sensed ECG data. The log of the ECG sensor system activity may include information indicating activation of a transmitter (e.g., the transceiver 346 of FIG. 3) of the ECG sensor system. The log of the ECG sensor system activity may include information indicating memory write activity of the ECG sensor system.

At 506, the method 500 may include sending one or more noise values to an automated troubleshooting system to troubleshoot the ECG sensor system. For example, the sensor system 320 of FIG. 3 may send the one or more noise values to the base system 430 of FIG. 4. The base system 430 may include an automated troubleshooting system. In some embodiments, at least a portion of the automated troubleshooting system may be included in the sensor system 320 for processing the one or more noise values.

Providing noise values to the automated troubleshooting system may enable the user of the sensor system to obtain troubleshooting information automatically based on the noise values. The noise values may provide an indication as to a potential non-heart beat source that may be affecting accuracy of the sensed ECG data.

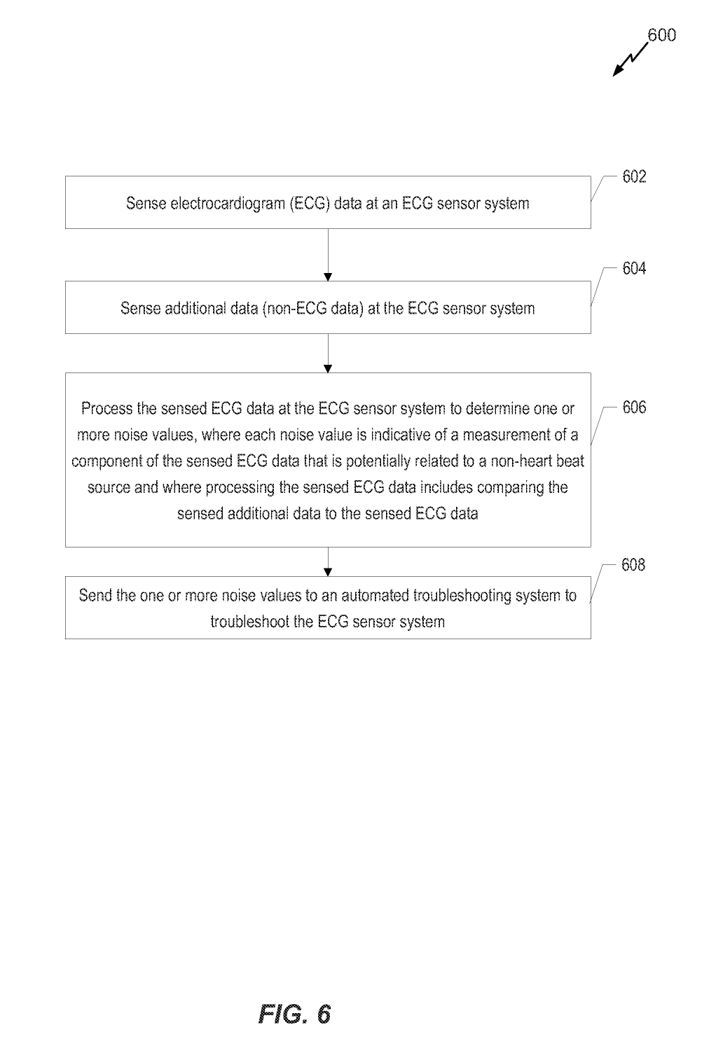

FIG. 6 is flow chart of a second particular embodiment of a method 600 performed at an ECG sensor system. For example, the method 600 may be performed by an ECG sensor system, such as the sensor system 120 of FIG. 1 or the sensor system 320 of FIG. 3.

At 602, the method 600 may include sensing ECG data at an ECG sensor system. For example, the sensor system 320 of FIG. 3 may sense the ECG data.

At 604, the method 600 may include sensing additional data at the ECG sensor system. The additional data may include sensed non-ECG data. For example, the sensor system 320 of FIG. 3 may sense the additional data (e.g., the non-ECG data) via the one or more non-ECG sensors 380. The non-ECG data may include a non-ECG signal that may correspond to one or more types of noise detectable via the non-ECG sensors 380. For example, one type of noise may include EMG noise that occurs based on electrical activity caused by muscle activity or movement within the patient's body. In this example, the non-ECG data may include an EMG signal that is indicative of time and intensity of muscle activity (e.g., muscle contraction). In another example, one type of noise may include power-line noise such as EMI. In this example, non-ECG data may include one or more measures of EMI, such as a signal-to-noise ratio (SNR) or a signal-to-interference (SIR) ratio.

At 606, the method 600 may include processing the sensed ECG data at the ECG sensor system to determine one or more noise values. Each of the one or more noise values may be indicative of a measurement of a component of the sensed ECG data that is potentially related to a non-heart beat source. For example, the processor 340 of FIG. 3 of sensor system 320 may process the sensed ECG data to determine the one or more noise values. The one or more noise values may correspond to one or more types of noise that may be detectable as a component of the sensed ECG data. The one or more types of noise may include power-line noise, baseline wander, EMG noise, electronics-related noise, other types of noise associated with one or particular artifacts, or a combination thereof. In some embodiments, the ECG data may be sent from the sensor system 320 to the base system 430 and the base system 430 may be configured to determine at least a portion of the one or more noise values.

Processing the sensed ECG data may include comparing the sensed additional data to the sensed ECG data. For example, the processor 340 may compare a portion of the sensed additional data, such as an EMG signal corresponding to sensed EMG data, to the sensed ECG data. By comparing the EMG signal to the sensed EMG data, the processor 340 can determine whether the sensed ECG data includes a component or attribute that corresponds to the EMG signal of the sensed additional data. In another example, the processor may compare a portion of the sensed additional data, such as a measure of EMI, to the sensed ECG data to determine whether the sensed ECG includes characteristics of the EMI measurement. The one or more noise values may be determined based on a determination that a portion of the sensed additional data is identified within the sensed ECG data. The one or more noise values may be determined based on the portion of the sensed additional data that matches the sensed ECG data.

At 608, the method 600 may include sending one or more noise values to an automated troubleshooting system to troubleshoot the ECG sensor system. For example, the sensor system 320 of FIG. 3 may send the one or more noise values to the base system 430 of FIG. 4. The base system 430 may include automated troubleshooting system. In some embodiments, at least a portion of the automated troubleshooting system may be included in the sensor system 320 for processing the one or more noise values.

By sensing additional data, such as non-ECG data, at the ECG sensor system, the ECG sensor system may increase certainty for determining one or more types of noise within the sensed ECG data. Identifying a presence of one or more types of noise with greater certainty enables the sensor system to isolate relevant sensed ECG data from other sensed ECG data that is based on one or more types of noise present in the sensor system during operation. Further, identification of relevant sensed ECG data enables the sensor system to more accurately identify seizure events that have occurred within the patient.

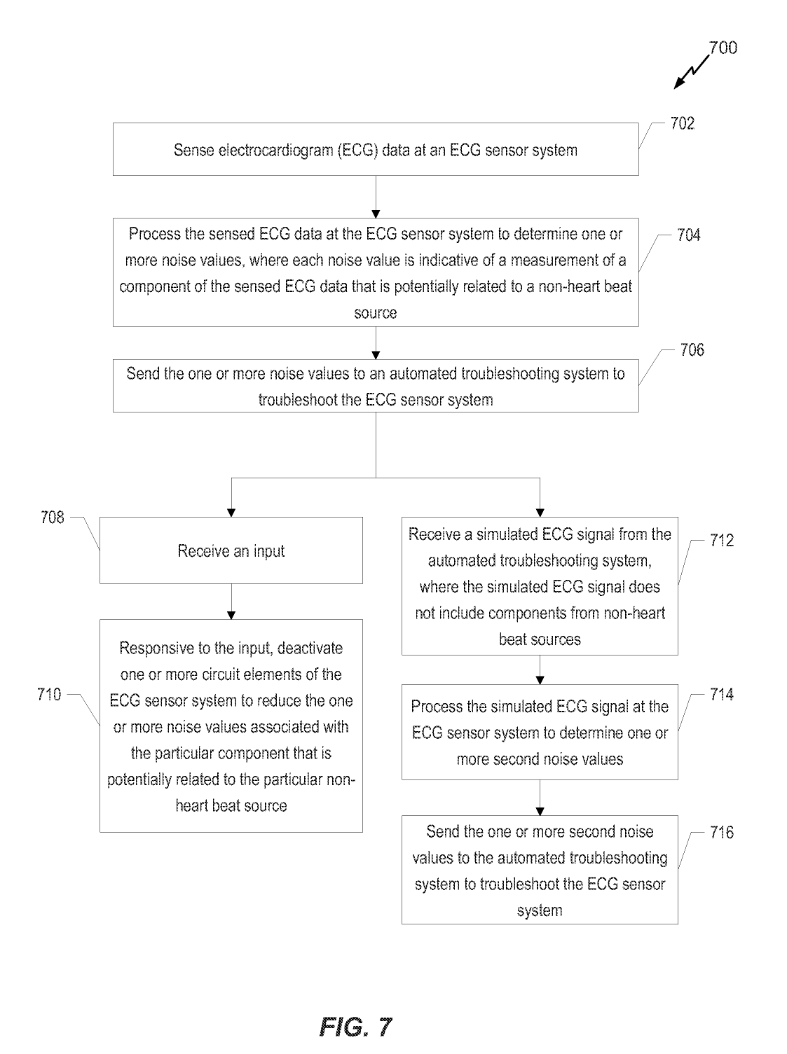

FIG. 7 is flow chart of a third particular embodiment of a method 700 performed at an ECG sensor system. For example, the method 700 may be performed by an ECG sensor system, such as the sensor system 120 of FIG. 1 or the sensor system 320 of FIG. 3.

At 702, the method 700 may include sensing ECG data at an ECG sensor system. For example, the sensor system 320 of FIG. 3 may sense ECG data.

At 704, the method 700 may include processing the sensed ECG data at the ECG sensor system to determine one or more noise values. The sensor system 320 of FIG. 3 may process the sensed ECG data to determine the one or more noise values. In some embodiments, the ECG data may be sent from the sensor system 320 to the base system 430 and the base system 430 may be configured to determine at least a portion of the one or more noise values.

At 706, the method 700 may include sending the one or more noise values to an automated troubleshooting system to troubleshoot the ECG sensor system. For example, the sensor system 320 of FIG. 3 may send one or more noise values to the base system 430 of FIG. 4. In this example, the base system 430 may include automated troubleshooting system. In some embodiments, at least a portion of the automated troubleshooting system may be included in the sensor system 320 for processing the one or more noise values.

In a particular embodiment, the automated troubleshooting system may perform an analysis of the one or more noise values. Based on the analysis, the automated troubleshooting system may identify a particular component of the sensed ECG data that is potentially related to a particular non-heart beat source. The analysis may include determining whether heart beat detection by the ECG sensor system satisfies one or more criteria.

In a particular embodiment, the automated troubleshooting system may provide an output indicating a suggested action to reduce the one or more noise values. The output may include an error code facilitating further troubleshooting or may include a failure code. The failure code may be usable to facilitate record keeping. The output may indicate that a particular portion of the ECG sensor system should be removed, replaced, or repaired. The output may be provided to a user, to a healthcare provider, to a manufacturer or a distributor of the ECG sensor system, to another party, or a combination thereof. In another particular embodiment, the output may be communicated to the ECG sensor system. The output may provide information to the ECG sensor system indicating one or more actions to be taken by the ECG sensor system to reduce the one or more noise values

At 708, the method 700 may include receiving an input at the ECG sensor system, such as the sensor system 320 of FIG. 3. In a particular embodiment, the input received by the ECG sensor system may be the output communicated by the automated troubleshooting system. The input may be received in response to sending the one or more noise values to the automated troubleshooting system at 706. In another particular embodiment, the input may be received at the ECG sensor system from a user (e.g., a patient) via a user input device, such as the user input device 360. The input may include information indicating one or more actions to be taken by the ECG sensor system to reduce the one or more noise values.

At 710, the method 700 may include deactivating, in response to the input, one or more circuit elements of the ECG sensor system to reduce the one or more noise values. For example, the sensor system 320 of FIG. 3, in response to the input (received at 708), may activate or deactivate one or more circuit elements of the sensor system 320 to reduce the one or more noise values. To illustrate, the sensor system 320 may deactivate the transceiver 346 to reduce power-line noise in response to receiving input indicating that activity of the transceiver 346 should be reduced. In another illustration, the sensor system 320 may deactivate the preprocessor 330 and the memory 350 to reduce electronics related noise caused by the preprocessor 330 and the memory 350, in response to input received from the automated troubleshooting system.

At 712, the method 700 may include receiving a simulated ECG signal from the automated troubleshooting system. In a particular embodiment, the simulated ECG signal does not include components from non-heart beat sources. For example, the sensor system 320 of FIG. 3 may receive a simulated ECG signal from the base system 430 of FIG. 4 (e.g., automated troubleshooting system). The simulated ECG signal may be based on previously detected heart rate information for the patient.

At 714, the method 700 may include processing the simulated ECG signal at the ECG sensor system to determine one or more second noise values. For example, the sensor system 320 of FIG. 3 may process the simulated ECG signal at the ECG sensor system to determine one or more second noise values. The one or more second noise values may be related to non-heart beat sources that may be present within the sensor system 320 or in an environment surrounding the sensor system 320, such that the one or more second noise values are introduced into the simulated ECG signal.

At 716, the method 700 includes sending the one or more second noise values to the automated troubleshooting system to troubleshoot the ECG sensor system. For example, the sensor system 320 of FIG. 3 may send the one or more second noise values to the base system 430 of FIG. 4 (the automated troubleshooting system) to troubleshoot the sensor system 320.

The automated troubleshooting system may analyze the one or more second noise values to determine whether the one or more second noise values correspond to one or more types of noise that may have been introduced into the simulated ECG signal. For example, the one or more types of noise may include power-line noise, baseline wander, EMG noise, electronics-related noise, other types of noise associated with one or particular artifacts, or a combination thereof. The automated troubleshooting system may perform troubleshooting by comparing the one or more second noise values to one or more criteria associated with a non-heart beat source. For example, the one or more criteria may include a threshold corresponding to a particular type of noise such that satisfying the threshold indicates the presence of the particular type of noise. Based on detecting the presence of the particular type of noise, the automated troubleshooting system may provide an output such as an error code identifying the particular type of noise or a troubleshooting procedure to reduce the particular type of noise.

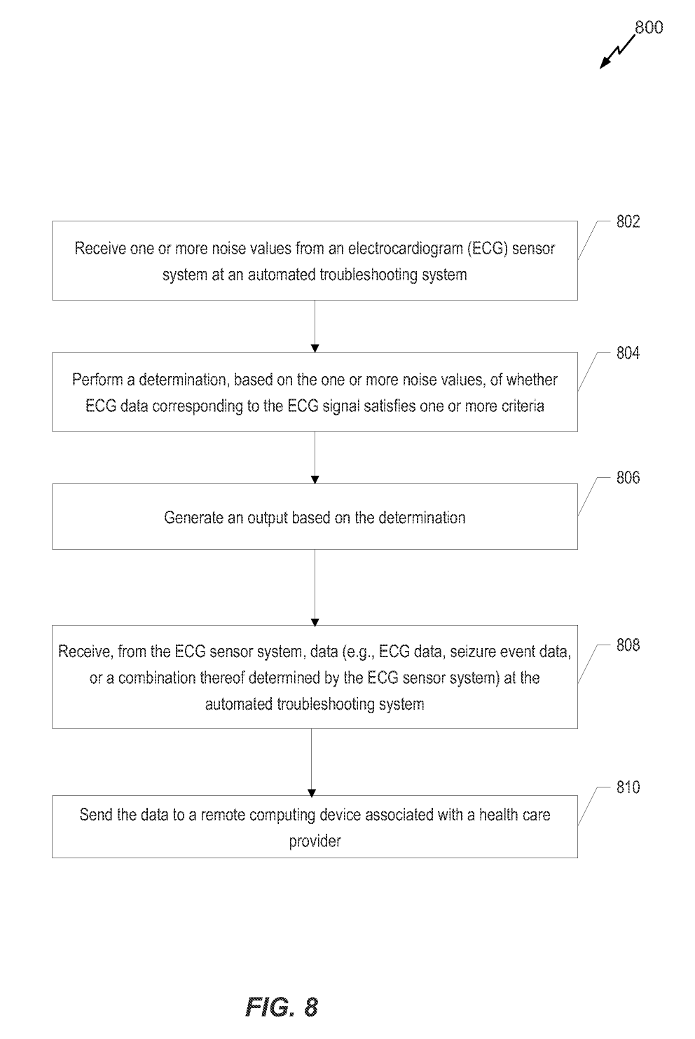

FIG. 8 is flow chart of a first particular embodiment of a method 800 performed at an automated troubleshooting system. The method 800 may be performed at the base system 130 of FIG. 1 or the base system 430 of FIG. 4.

At 802, the method 800 includes receiving one or more noise values from an ECG sensor system at the automated troubleshooting system. Each noise value may be indicative of a measurement of a component of an ECG signal sensed by the ECG sensor system. The component of the ECG signal may be related to a non-heart beat source. For example, the base system 430 of FIG. 4 may receive one or more noise values from the ECG sensor system 320 of FIG. 3. In a particular embodiment, the one or more noise values may be received in a predetermined format such as a vector format, in which each of the one or more noise values are arranged in a predetermined order. For example, the base system may receive a vector of data in which a first entry corresponds to a first type of noise value, a second entry corresponds to a second type of noise value, a third entry corresponds to a third type of noise value, etc.

At 804, the method 800 includes performing a determination, based on the one or more noise values, of whether ECG data corresponding to the sensed ECG signal satisfies one or more criteria. For example, the base system 430 of FIG. 4 may determine, based on the one or more noise values, whether the ECG data corresponding to the sensed ECG signal satisfies one or more criteria. The one or more criteria may be defined based on one or more threshold ranges corresponding to values that are associated with the one or more type of noise that may be present in an ECG signal and that are attributable to a particular non-heart beat source (e.g., components of the sensor system 320 of FIG. 3, movement activity within the patient's body, power-line transmission). The one or more types of noise may include power-line noise, baseline wander, EMG noise, electronics-related noise, other types of noise associated with one or particular artifacts, or a combination thereof. In some embodiments, at least a portion of the automated troubleshooting system may be included in the sensor system 320 for processing the one or more noise values.

At 806, the method 800 includes generating an output based on the determination. For example, the base system 430 of FIG. 4 may generate an output based on the determination of whether the ECG data corresponding to the sensed ECG signal satisfies one or more criteria. In a particular embodiment, the output may include a recommended troubleshooting procedure. In another particular embodiment, the output may include diagnostic information (e.g., an error code), which may facilitate further troubleshooting.

The output generated by the automated troubleshooting system may provide a user of the ECG sensor system with information to adjust operation of the ECG sensor system to improve accuracy of the sensed ECG data. For example, the output may indicate that the ECG sensor system should be relocated to a new location, such as another room, because the sensed ECG data may affected, at the current location, by a non-heart beat source that introduces noise to the sensed ECG data. In another example, the output may indicate that a patch of the ECG sensor system should be repositioned on the patient's body to improve the accuracy of the sensed ECG data. Thus, the user may save time by avoiding efforts to contact a technician or a manufacturer to determine how to adjust or where to relocate the ECG sensor system. The troubleshooting information provided by the automated troubleshooting system may offer greater reliability because the output is based on a determination using data from actual usage of the ECG sensor system rather than usage based on test performed outside (e.g., repair lab) the user's environment.

At 808, the method 800 includes receiving, from the ECG sensor system, data (e.g., ECG data, seizure event data, or a combination thereof) at the automated troubleshooting system. For example, the base system 430 of FIG. 4 may receive sensed ECG data from the sensor system 320 of FIG. 3. In another example, the base system 430 may receive seizure event data from the sensor system 320. The seizure event data may have been determined by the sensor system 320 based on the ECG data sensed by the sensor system 320.

At 810, the method 800 includes sending the data (e.g., the ECG data, the seizure event data, or a combination thereof) to a remote computing device associated with a health care provider. For example, the base system 430 of FIG. 4 may send the data to the remote computing device 140 of FIG. 1 associated with a health care provider.

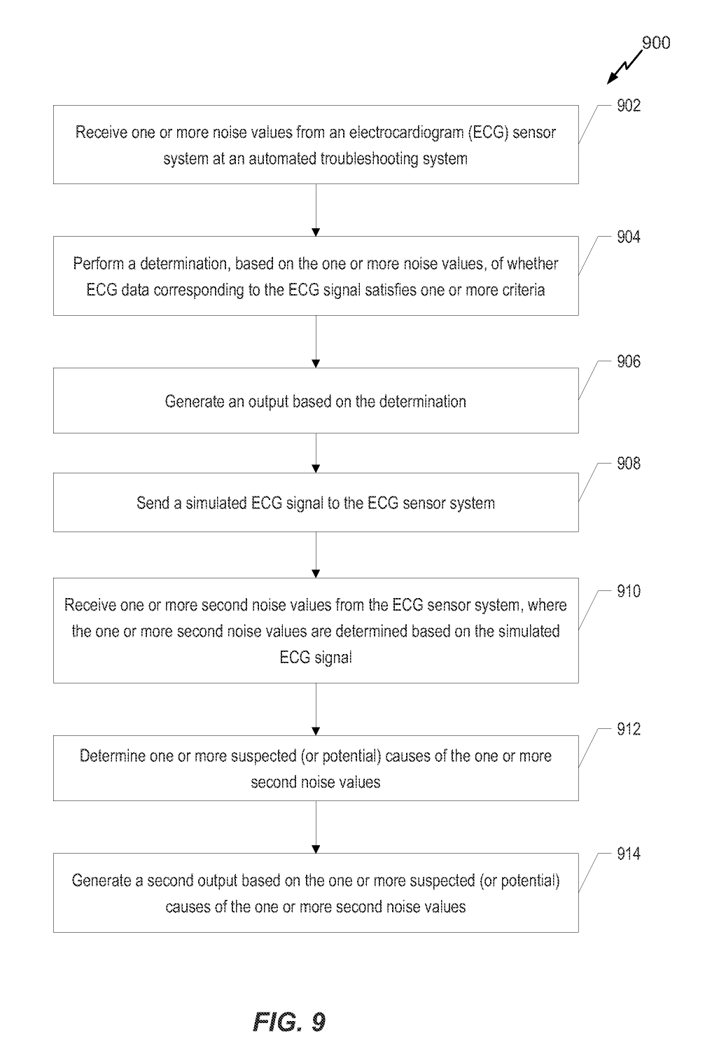

FIG. 9 is flow chart of a second particular embodiment of a method 900 performed at an automated troubleshooting system. For example, the method 900 may be performed at the base system 130 of FIG. 1 or the base system 430 of FIG. 4.

At 902, the method 900 includes receiving one or more noise values from an ECG sensor system at the automated troubleshooting system. Each noise value may be indicative of a measurement of a component of an ECG signal sensed by the ECG sensor system. The measurement of the component may be potentially related to a non-heart beat source. For example, the base system 430 of FIG. 4 may receive the one or more noise values from the sensor system 320 of FIG. 3.

At 904, the method 900 includes performing a determination, based on the one or more noise values, of whether ECG data corresponding to the sensed ECG signal satisfies one or more criteria. For example, the base system 430 of FIG. 4 may determine, based on the one or more noise values, whether the ECG data corresponding to the sensed ECG signal satisfies one or more criteria. The one or more criteria may be defined based on one or more threshold ranges corresponding to the one or more type of noise present in an ECG signal and that are attributable to a particular non-heart beat source (e.g., components of the sensor system 320 of FIG. 3, movement activity within the patient's body, power-line transmission). The one or more types of noise may include power-line noise, baseline wander, EMG noise, electronics-related noise, other types of noise associated with one or particular artifacts, or a combination thereof. In some embodiments, at least a portion of the automated troubleshooting system may be included in the sensor system 320 for processing the one or more noise values.

At 906, the method 900 includes generating an output based on the determination. For example, the base system 430 of FIG. 4 may generate an output based on the determination of whether the ECG data corresponding to the sensed ECG signal satisfies one or more criteria. In a particular embodiment, the output may include a recommended troubleshooting procedure. In another particular embodiment, the output may include diagnostic information (e.g., an error code), which may facilitate further troubleshooting.

At 908, the method 900 includes sending a simulated ECG signal to the ECG sensor system. For example, the base system 430 of FIG. 4 may send a simulated ECG signal to the sensor system 320 of FIG. 3. The automated troubleshooting system may send the simulated ECG signal to the ECG sensor system during a test mode of operation. The simulated ECG signal may be an ECG signal corresponding to a normal ECG signal of a patient based on historical medical records. The normal ECG signal may be associated with a patient using the ECG sensor system. The simulated ECG signal may be sent to the ECG sensor system to diagnose a problem at the sensor system, which may be related to one or more types of noise occurring at the ECG sensor system.

At 910, the method 900 includes receiving one or more second noise values from the ECG sensor system, where the one or more second noise values are determined based on the simulated ECG signal. For example, the base system 430 of FIG. 4 may receive one or more second noise values from the ECG sensor system 320 of FIG. 3. The ECG sensor system may determine the one or more second noise values based on the simulated ECG signal. The one or more second noise values may be indicative of a measurement of a component of the simulated ECG signal that is potentially related to a non-heart beat source. The one or more second noise values may correspond to one or more types of noise. The one or more types of noise may include power-line noise, baseline wander, EMG noise, electronics-related noise, other types of noise associated with one or particular artifacts, or a combination thereof.

At 912, the method 900 includes determining one or more suspected or potential causes of the one or more second noise values. For example, the base system 430 of FIG. 4 may determine one or more suspected or potential causes of the one or more second noise values. Determining the one or more suspected or potential causes may include using a SVM to apply a classification algorithm to determine whether the one or more second noise values satisfy one or more criteria. For example, the base system 430 may use the SVM 436 to determine whether the one or more second noise values satisfy one or more criteria. The classification algorithm may be associated with a supervised learning model that is defined based on the one or more criteria. The one or more criteria may correspond to one or more threshold value ranges that correspond to one or more types of noise (e.g., power-line noise, baseline wander, electronics-related noise, and EMG noise). Application of the classification algorithm to the one or more second noise values may produce a result that identifies the one or more types of noise, which may be used to determine the one or more suspected causes. For example, the electronics-related noise may indicate that a suspected cause of one of the one or more second noise values is a component of the sensor system 420.

At 914, the method 900 includes generating a second output based on the one or more suspected or potential causes of the one or more second noise values. For example, the base system 430 of FIG. 4, may generate an output (e.g., second output) based on the one or more suspected or potential causes of the one or more second noise values.

Although the description above contains many specificities, these specificities are utilized to illustrate some particular embodiments of the disclosure and should not be construed as limiting the scope of the disclosure. The scope of this disclosure should be determined by the claims and their legal equivalents. A method or device does not have to address each and every problem to be encompassed by the present disclosure. All structural, chemical and functional equivalents to the elements of the disclosure that are known to those of ordinary skill in the art are expressly incorporated herein by reference and are intended to be encompassed by the present claims. A reference to an element in the singular is not intended to mean one and only one, unless explicitly so stated, but rather it should be construed to mean at least one. No claim element herein is to be construed under the provisions of 35 U.S.C. §112, sixth paragraph, unless the element is expressly recited using the phrase “means for.” Furthermore, no element, component or method step in the present disclosure is intended to be dedicated to the public, regardless of whether the element, component or method step is explicitly recited in the claims.

The disclosure is described above with reference to drawings. These drawings illustrate certain details of specific embodiments of the systems and methods and programs of the present disclosure. However, describing the disclosure with drawings should not be construed as imposing on the disclosure any limitations that may be present in the drawings. The present disclosure contemplates methods, systems and program products on any machine-readable media for accomplishing its operations. The embodiments of the present disclosure may be implemented using an existing computer processor, a special purpose computer processor, or by a hardwired system.

As noted above, embodiments within the scope of the present disclosure include program products including machine-readable media for carrying or having machine-executable instructions or data structures stored thereon. Such machine-readable media can be any available media which can be accessed by a general purpose or special purpose computer or other machine with a processor. By way of example, such machine-readable media can include RAM, ROM, EPROM, EEPROM, CD ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. The disclosure may be utilized in a non-transitory media. When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general purpose computer, a special purpose computer, or special purpose processing machines to perform a certain function or group of functions.

Embodiments of the disclosure are described in the general context of method steps which may be implemented in one embodiment by a program product including machine-executable instructions, such as program code, for example, in the form of program modules executed by machines in networked environments. Generally, program modules include routines, programs, objects, components, data structures, etc., that perform particular tasks or implement particular abstract data types. Machine-executable instructions, associated data structures, and program modules represent examples of program code for executing steps of the methods disclosed herein. The particular sequence of such executable instructions or associated data structures represent examples of corresponding acts for implementing the functions described in such steps.

Embodiments of the present disclosure may be practiced in a networked environment using logical connections to one or more remote computers having processors. Logical connections may include a local area network (LAN) and a wide area network (WAN) that are presented here by way of example and not limitation. Such networking environments are commonplace in office-wide or enterprise-wide computer networks, intranets and the Internet and may use a wide variety of different communication protocols. Those skilled in the art will appreciate that such network computing environments will typically encompass many types of computer system configurations, including personal computers, hand-held devices, multi-processor systems, microprocessor-based or programmable consumer electronics, network PCs, servers, minicomputers, mainframe computers, and the like. For example, the network computing environment may include the sensor system 120 of FIG. 1, the base system 130, the remote computing device 140, the sensor system 320 of FIG. 3, the base system 430 of FIG. 4, the sensor system 420, the mobile computing device 402, the computing device 404, or any combination thereof. Embodiments of the disclosure may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

An exemplary system for implementing the overall system or portions of the disclosure might include a general purpose computing device in the form of a computer, including a processing unit, a system memory, and a system bus that couples various system components including the system memory to the processing unit. For example, the general purpose computing device may include the sensor system 120 of FIG. 1, the base system 130, the remote computing device 140, the sensor system 320 of FIG. 3, the base system 430 of FIG. 4, the sensor system 420, the mobile computing device 402, the computing device 404. The system memory may include read only memory (ROM) and random access memory (RAM). The computer may also include a magnetic hard disk drive for reading from and writing to a magnetic hard disk, a magnetic disk drive for reading from or writing to a removable magnetic disk, and an optical disk drive for reading from or writing to a removable optical disk such as a CD ROM or other optical media. The drives and their associated machine-readable media provide nonvolatile storage of machine-executable instructions, data structures, program modules, and other data for the computer.

It should be noted that although the flowcharts provided herein show a specific order of method steps, it is understood that the order of these steps may differ from what is depicted. Also two or more steps may be performed concurrently or with partial concurrence. Such variation will depend on the software and hardware systems chosen and on designer choice. It is understood that all such variations are within the scope of the disclosure.

The foregoing description of embodiments of the disclosure has been presented for purposes of illustration and description. It is not intended to be exhaustive or to limit the disclosure to the precise form disclosed, and modifications and variations are possible in light of the above teachings or may be acquired from practice of the disclosure. The embodiments were chosen and described in order to explain the principals of the disclosure and its practical application to enable one skilled in the art to utilize the disclosure in various embodiments and with various modifications as are suited to the particular use contemplated.

The Abstract of the Disclosure is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, the claimed subject matter may be directed to less than all of the features of any of the disclosed embodiments.

What is claimed is:

1. A method comprising:
sensing electrocardiogram (ECG) data at an ECG sensor system;
processing the sensed ECG data at the ECG sensor system to determine one or more noise values, wherein each noise value is indicative of a measurement of a component of the sensed ECG data that is potentially related to a non-heart beat source; and
sending the one or more noise values to an automated troubleshooting system to troubleshoot the ECG sensor system.

2. The method of claim 1, wherein the automated troubleshooting system performs an analysis of the one or more noise values and identifies a source of a particular component of the sensed ECG data that is related to a particular non-heart beat source based on the analysis.

3. The method of claim 2, further comprising, after sending the one or more noise values, receiving an input and, responsive to the input, deactivating one or more circuit elements of the ECG sensor system to reduce the one or more noise values associated with the particular component that is related to the particular non-heart beat source.

4. The method of claim 1, wherein processing the sensed ECG data to determine the one or more noise values includes generating an estimate of power-line noise, generating an estimate of baseline wander, generating an estimate of electromyographic (EMG) noise, generating an estimate of electronics noise of the ECG sensor system, or a combination thereof.

5. The method of claim 4, further comprising receiving movement data from an accelerometer and generating the estimate of the EMG noise by comparing the movement data and the sensed ECG data.

6. The method of claim 4, wherein generating the estimate of the electronics noise of the ECG sensor system includes applying a wavelet filter to detect a pattern in a sample portion of the sensed ECG data.

7. The method of claim 4, wherein generating the estimate of the electronics noise of the ECG sensor system includes applying a filter that is adapted to pass predetermined electronic signal artifacts within a sample portion of the sensed ECG data, wherein the predetermined electronic signal artifacts correspond to signal artifacts that are known to be generated by the ECG sensor system.

8. The method of claim 4, further comprising comparing a sample portion of the sensed ECG data to a log of ECG sensor system activity to identify signal artifacts generated by the ECG sensor system, wherein the log of ECG sensor system activity includes at least one of information indicating activation of a transmitter of the ECG sensor system, information indicating memory write activity of the ECG sensor system.

9. The method of claim 1, further comprising sensing additional data at the ECG sensor system, wherein the additional data is non-ECG data, wherein processing the sensed ECG data to determine the one or more noise values includes comparing the sensed additional data to the sensed ECG data.

10. The method of claim 1, further comprising after sending the one or more noise values to the automated troubleshooting system:
receiving a simulated ECG signal from the automated troubleshooting system, wherein the simulated ECG signal does not include components from non-heart beat sources;
processing the simulated ECG signal at the ECG sensor system to determine one or more second noise values; and
sending the one or more second noise values to the automated troubleshooting system to troubleshoot the ECG sensor system.

11. An electrocardiogram (ECG) sensor system comprising:
one or more interface connectors configured to be coupled to one or more electrodes; and
a processor configured to receive an ECG signal via the one or more interface connectors, the processor configured to determine one or more noise values associated with the ECG signal, wherein each noise value is indicative of a measurement of a component of the ECG signal that is potentially related to a non-heart beat source, the processor configured to send the one or more noise values to an automated troubleshooting system.

12. The ECG sensor system of claim 11, further comprising a transmitter coupled to the processor, the transmitter configured to send the one or more noise values to the automated troubleshooting system.

13. The ECG sensor system of claim 11, further comprising a preprocessor coupled to the one or more interface connectors, the preprocessor configured to amplify the ECG signal received via the interface connectors and to provide the amplified ECG signal to the processor, the preprocessor further configured to perform heart beat detection based on the ECG signal.

14. The ECG sensor system of claim 13, further comprising a memory coupled to the processor, wherein the processor is further configured to analyze output of the preprocessor to detect a potential seizure event and to log the potential seizure event in the memory.

15. The ECG sensor system of claim 13, further comprising a housing at least partially enclosing the one or more interface connectors, the preprocessor, and the processor.

16. The ECG sensor system of claim 11, further comprising a patch that includes the one or more electrodes on a first side of the patch and that includes one or more patch interface connectors on a second side of the patch, the one or more patch interface connectors corresponding to the one or more interface connectors.

17. The ECG sensor system of claim 11, further comprising one or more non-ECG sensors, wherein the transmitter is further operable to send non-heart beat data received from the one or more non-ECG sensors to the automated troubleshooting system.

18. A computer-readable storage medium including instructions that, when executed by a processor, cause the processor to:
sense electrocardiogram (ECG) data at an ECG sensor system;
process the sensed ECG data at the ECG sensor system to determine one or more noise values, wherein each noise value is indicative of a measurement of a component of the sensed ECG data that is potentially related to a non-heart beat source; and
send the one or more noise values to an automated troubleshooting system to troubleshoot the ECG sensor system.

19. The computer-readable storage medium of claim 18, further comprising instructions that, when executed by a processor, cause the processor to:
receive a simulated ECG signal from the automated troubleshooting system after sending the one or more noise values to an automated troubleshooting system, wherein the simulated ECG signal does not include components from non-heart beat sources;
process the simulated ECG signal at the ECG sensor system to determine one or more second noise values; and
send the one or more second noise values to the automated troubleshooting system to troubleshoot the ECG sensor system.

20. The computer-readable storage medium of claim 18, wherein the instructions that cause the processor to process the sensed ECG data to determine one or more noise values further comprises instructions that, when executed by a processor, cause the processor to generate one of an estimate of power-line noise, an estimate of baseline wander, an estimate of electromyographic (EMG) noise, an estimate of electronics noise of the ECG sensor system, or a combination thereof.

21. The computer-readable storage medium of claim 18, further comprising instructions that, when executed by a processor, cause the processor to:
sense additional data at the ECG sensor system, wherein the additional data is non-ECG data; and
compare the sensed additional data to the sensed ECG data to determine the one or more noise values.
